(19) United States
(12) Patent Application Publication
(10) Pub. No.: US 2021/0401952 A1
(43) Pub. Date: Dec. 30, 2021

(54) Method of treating AML subtypes using arginine-depleting agents

(71) Applicant: Vision Global Holdings Limited, Hong Kong (CN)

(72) Inventors: Sui-Yi KWOK, Hong Kong (CN); Norman Fung-Man WAI, Vancouver (CA); Terence Shau Yin WAI, Vancouver (CA); Yun Chung LEUNG, Hong Kong (CN)

(21) Appl. No.: 16/946,590

(22) Filed: Jun. 29, 2020

(51) Int. Cl. A61K 38/50; A61K 38/44; A61K 38/51; A61K 9/00; A61P 35/00

(52) U.S. Cl. CPC A61K 38/50; A61K 38/44; A61K 38/51; A61K 9/0019; C12Y 113/12001; C12Y 305/03006; C12Y 305/03001; C12Y 401/01019; A61P 35/00

(57) ABSTRACT

The invention provides a method for treating acute myeloid leukemia (AML) in a subject in need thereof, said method comprising administering a therapeutically effective amount of an arginine-depleting agent to the subject, wherein the AML is of the French-American-British (FAB) subtype M0 (undifferentiated acute myeloblastic leukemia), M2 (acute myeloblastic leukemia with maturation), M4 (acute myeloblastic leukemia with maturation), M4 eos (acute myelomonocytic leukemia with eosinophilia), M5 (acute monocytic leukemia), M6 (acute erythroid leukemia) or M7 (acute megakaryoblastic leukemia).

### TECHNICAL FIELD

The present invention relates generally to the fields of biology and medicine, and more specifically to methods for treating acute myeloid leukemia (AML). Still more specifically, the present invention relates to methods for treating AML subtypes using arginine-depleting agents and the use of arginine-depleting agents in the manufacture of medicaments for the treatment of AML.

### BACKGROUND

The following discussion of the background of the invention is merely provided to aid the reader in understanding the invention, and is not admitted to describe or constitute prior art to the invention.

Acute myeloid leukemia (AML), also known as acute myelogenous leukemia, is a genetically heterogeneous aggressive cancer in which the accumulation of genetic alterations results in uncontrolled clonal proliferation of myeloid progenitor cells in the bone marrow and blood. More severe cases involve the infiltration of organs by the abnormal cells. AML is one of the most common acute leukemias in adults and children, accounting for approximately 80% of adult cases and approximately 20% of childhood leukemia diagnoses. Most cases of AML occur in adults, with the average age of diagnosis being 68 years. The five-year survival rate for people over the age of 20 diagnosed with AML is only 25%.

AML is a heterogeneous disease which is classified into several subtypes. There are two major classification systems for AML subtypes—the French-American-British (FAB) system and the World Health Organization (WHO) classification system. The FAB classification system is the one most commonly used and is the one referred to herein. Most people diagnosed with AML have one of nine different FAB subtypes of AML (M0, M1, M2, M3, M4, M4 eos, M5, M6 & M7). The prognosis of a case of AML is often dependent, inter alia, on the FAB AML subtype.

Despite technological advances and an emerging understanding of the disease, overall survival rates of patients diagnosed with AML have plateaued and people continue to die of the disease in significant numbers. Chemotherapy is currently the main mode of treatment for AML and includes two main phases: induction and consolidation. Induction therapy aims for complete remission of the cancer. Consolidation is a term given to post-remission therapy. Patients may die within a few days of the commencement of treatment due to treatment-related mortality. The major reason patients are not cured is resistance to treatment, which often manifests as a relapse from remission. However, there is no current standard of care for adult relapsed or refractory AML, and the prognosis in such patients is generally poor.

AML remains a challenging illness and a need exists for new therapeutic approaches for the treatment of this aggressive cancer.

### SUMMARY

The present invention provides methods for treating acute myeloid leukemia (AML) using arginine-depleting agents and the use of arginine-depleting agents in the manufacture of medicaments for the treatment of AML.

The inventors of the present invention have surprisingly found that certain FAB AML subtypes respond markedly better to arginine depletion than others. The methods and uses described herein may therefore be useful for targeted treatment of AML based on FAB AML subtype.

In a first aspect, the present invention provides a method for treating acute myeloid leukemia (AML) in a subject in need thereof, said method comprising administering a therapeutically effective amount of an arginine-depleting agent to the subject, wherein the AML is of the French-American-British (FAB) subtype M0 (undifferentiated acute myeloblastic leukemia), M2 (acute myeloblastic leukemia with maturation), M4 (acute myeloblastic leukemia with maturation), M4 eos (acute myelomonocytic leukemia with eosinophilia), M5 (acute monocytic leukemia), M6 (acute erythroid leukemia) or M7 (acute megakaryoblastic leukemia).

In one embodiment of the first aspect, the arginine-depleting agent comprises an arginine catabolic enzyme.

In one embodiment of the first aspect, the arginine catabolic enzyme is an arginine deiminase, arginase, arginine decarboxylase or arginine 2-monooxygenase.

In one embodiment of the first aspect, the arginine-depleting agent is a synthetic arginine-depleting agent.

In one embodiment of the first aspect, the arginine-depleting agent comprises human serum albumin, an albumin binding domain, an Fe region of an immunoglobulin, a polyethylene glycol (PEG) group, human transferrin, XTEN, a proline-alanine-serine polymer (PAS), an elastin-like polypeptide (ELP), a homo-amino-acid polymer (HAP), artificial gelatin-like protein (GLK), a carboxy-terminal peptide (CTP), or a combination thereof.

In one embodiment of the first aspect, the arginine-depleting agent comprises human serum albumin, an albumin binding domain, or a combination thereof.

In one embodiment of the first aspect, the arginine-depleting agent comprises or consists of an amino acid sequence having at least 95%, 96%, 97%, 98% or 99% sequence identity to SEQ ID NO: 1.

In one embodiment of the first aspect, the arginine-depleting agent comprises or consists of an amino acid sequence as defined in SEQ ID NO: 1.

In one embodiment of the first aspect, the AML is of the FAB subtype M4 or M7.

In one embodiment of the first aspect, the AML is of the FAB subtype M7 and the arginine-depleting agent comprises or consists of an amino acid sequence having at least 95%, 96%, 97%, 98% or 99% sequence identity to SEQ ID NO: 1.

In one embodiment of the first aspect, the AML is of the FAB subtype M7 and the arginine-depleting agent comprises or consists of an amino acid sequence as defined in SEQ ID NO: 1.

In one embodiment of the first aspect, the AML is auxotrophic for arginine.

In one embodiment of the first aspect, the arginine-depleting agent is administered intramuscularly, intravenously, subcutaneously or orally.

In one embodiment of the first aspect, the arginine-depleting agent is administered intravenously.

In one embodiment of the first aspect, the subject is human.

### Definitions

Certain terms are used herein which shall have the meanings set forth as follows.

As used in this application, the singular form “a”, “an” and “the” include plural references unless the context clearly dictates otherwise.

As used herein, the term “comprising” means “including”, in a non-exhaustive sense. Variations of the word “comprising”, such as “comprise” and “comprises” have correspondingly varied meanings.

As used herein, the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any numerical value derivable therein, and any range derivable therein.

As used herein, the term “between” when used in reference to a range of numerical values encompasses the numerical values at each endpoint of the range.

As used herein, the term “synthetic”, when used to describe a product, refers to a product produced by human agency as opposed to a naturally occurring product. For example, a “synthetic” arginine-depleting agent refers to an arginine-depleting agent which has been produced by artificial chemical reactions.

As used herein, the terms “treat”, “treating”, “treatment”, and the like refer to reducing or ameliorating a disorder/disease and/or symptoms associated therewith. It will be appreciated, although not precluded, that treating a disorder or condition does not require that the disorder, condition, or symptoms associated therewith be completely eliminated.

As used herein, the term “catabolism” or “catabolic” refers to a chemical reaction in which a molecule decomposes into other, e.g., smaller, molecules. For example, the term “arginine catabolic enzyme” includes any enzyme capable of reacting with arginine thereby transforming it into other molecules, such as ornithine, citrulline, and agmatine.

As used herein, the term “subject” refers to any animal (e.g., a mammal), including, but not limited to, humans, non-human primates, canines, felines, and rodents.

As used herein, the terms “FAB subtype”, “FAB AML subtype” and “FAB AML” refer to a subtype of AML as classified by the French-American-British (FAB) classification system. The FAB system divides AML into nine subtypes, M0, M1, M2, M3, M4, M4 eos, M5, M6 and M7, based on the type of cell the leukemia develops from and how mature the cells are.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences being compared. For example, a nucleotide sequence (“subject sequence”) having at least 95% “sequence identity” with another nucleotide sequence (“query sequence”) is intended to mean that the subject sequence is identical to the query sequence except that the subject sequence may include up to five nucleotide alterations per 100 nucleotides of the query sequence. In other words, to obtain a nucleotide sequence of at least 95% sequence identity to a query sequence, up to 5% (i.e. 5 in 100) of the nucleotides in the subject sequence may be inserted or substituted with another nucleotide or deleted.

As used herein, the term “auxotrophic”, when used to describe a cancer, refers to a cancer which is unable to synthesize one or more specific substances required for growth and/or metabolism. For example, an AML which is “auxotrophic for arginine” refers to an AML which is unable to synthesize arginine.

### BRIEF DESCRIPTION OF THE DRAWINGS

The above and other aspects and embodiments of the present disclosure will become apparent from the following description of the disclosure, when taken in conjunction with the accompanying drawings, in which:

FIG. 1 provides a schematic of the relationship between argininosuccinate synthetase (ASS) and the urea cycle.

FIG. 2 is an image of a gel showing expression levels of autophagic (LC3-II, BECLIN-1 and phospho-AMPKα) and apoptotic (PARP-1) markers in a pancreatic cell line (Mia PaCa-2) after NEI-01 treatment.

FIG. 3 is an image of a gel showing expression levels of autophagic (LC3-II, p62, phospho-AMPK-α, AMPK-α) and apoptotic (Caspase-9) markers in an AML cell line (HL-60) after NEI-01 treatment.

FIG. 4 provides Kaplan-Meier survival curves of mice with AML in a C1498 (M4) syngeneic AML model.

FIG. 5A provides images of the tumour burden monitored and quantified using in vivo bioluminescence imaging in a C1498 (M4) syngeneic AML model.

FIG. 5B provides images of the tumour burden monitored and quantified using in vivo bioluminescence imaging in a C1498 (M4) syngeneic AML model.

FIG. 5C provides images of the tumour burden monitored and quantified using in vivo bioluminescence imaging in a C1498 (M4) syngeneic AML model.

FIG. 5D provides images of the tumour burden monitored and quantified using in vivo bioluminescence imaging in a C1498 (M4) syngeneic AML model.

FIG. 6 provides a graph of the tumour burden monitored and quantified using in vivo bioluminescence imaging in a C1498 (M4) syngeneic AML model.

FIG. 7 provides graphs showing the inhibition of tumour growth following repeated NEI-01 treatments in a KG-1-Derived Acute Myeloid Leukaemia (FAB AML M0) Xenograft Model. (A) shows the change in average tumour volume in 4 weeks. (B) shows the change in average T/C % over 4 weeks. Tumour volume was measured every 3 days. By day 28, a 39% reduction was observed in the treatment group. Statistics were calculated using RM two-way ANOVA, followed by Sidak's multiple comparison for post-hoc analysis. ** indicates a p-value of less than 0.01. *** indicates a p-value of less than 0.001.

FIG. 8 provides a graph showing bioluminescence signals in mice transplanted with HL-60-gfphi-Luc+ AML cells. In vivo BLI was performed twice a week and the changes in BLI intensity were plotted. Data are expressed as mean±SEM.

FIG. 9 provides graphs which show the inhibition of tumour growth following repeated NEI-01 treatments in a P31/FUJ-Derived Acute Myeloid Leukaemia (FAB AML M5) Xenograft Model. (A) shows the change in average tumour volume in 4 weeks. (B) shows the change in average T/C % over 4 weeks. Statistics were calculated using RM two-way ANOVA, followed by Sidak's multiple comparison for post-hoc analysis. * indicates a p-value of less than 0.05. ** indicates a p-value of less than 0.01. *** indicates a p-value of less than 0.001. (C) shows the difference in tumour weight between control and treated groups.

FIG. 10 provides graphs which show the inhibition of tumour growth following repeated NEI-01 treatments in a MKPL-1-Derived Acute Myeloid Leukaemia (FAB AML M7) Xenograft Model. (A) shows the change in average tumor volume in 3 weeks. (B) provides the change in average T/C % over 3 weeks. (C) shows the tumor weight after the termination of the study. Statistics were calculated using RM two-way ANOVA, followed by Sidak's multiple comparison for post-hoc analysis. * indicates a p-value of less than 0.05. *** indicates a p-value of less than 0.001.

FIG. 11 provides a graph of growth curves of tumour burden (presented by % of hCD45+ cells) in peripheral blood. Data is expressed as mean±SEM.

FIG. 12 is a graph of Kaplan-Meirer survival curves of mice in an AM8096 model.

FIG. 13 provides graphs showing the in vivo response of Jurkat cells to NEI-01. (A) shows the change in tumour volume (%) in 4 weeks. (B) shows the change in average T/C % in 4 weeks. Tumour volume was measured twice a week. Data are expressed as mean±SEM. A two-tailed student T-test was used. * indicates a p value of equal to or less than 0.05.

FIG. 14A provides graphs of the mean plasma concentration of NEI-01 in male mice and for a Repeated Dose Study on Day 1 and Day 22.

FIG. 14B provides graphs of the mean plasma concentration of NEI-01 in female mice for a Repeated Dose Study on Day 1 and Day 22.

### DETAILED DESCRIPTION

Acute myeloid leukemia (AML) is one of the most common acute leukemias in adults and children. Current methods for treating AML are sometimes responsible for treatment-related mortality. In cases where treatment achieves initial success, relapse from remission is common. It is highly important that new therapeutic approaches are developed for this aggressive cancer.

The present invention provides methods for treating AML using arginine-depleting agents. The methods provided herein may reduce or ameliorate the disease and/or symptoms associated therewith. The methods may or may not completely eliminate said disease and/or symptoms. The arginine-depleting agents described herein may also be used for the manufacture of medicaments for the treatment of AML, which may reduce or ameliorate the disease and/or symptoms associated therewith, and may or may not completely eliminate said disease and/or symptoms.

FAB AML Subtypes

AML is a heterogeneous disease which is classified into several subtypes. There are two major classification systems for AML subtypes—the French-American-British (FAB) system and the World Health Organization (WHO) classification system. The FAB classification system is the one most commonly used and is the one referred to herein. Most people diagnosed with AML have one of nine different FAB subtypes of AML (M0, M1, M2, M3, M4, M4 eos, M5, M6 & M7). The prognosis of a case of AML is often dependent, inter alia, on the AML subtype.

The FAB classification system divides AML into subtypes M0 to M7 based on the type of cell the leukemia develops from and how mature the cells are, as outlined in Table 1:

TABLE 1
FAB AML subtypes and descriptions

| FAB subtype | Description |
|---|---|
| M0 | Undifferentiated acute myeloblastic leukemia |
| M1 | Acute myeloblastic leukemia with minimal |
| | maturation |
| M2 | Acute myeloblastic leukemia with maturation |
| M3 | Acute promyelocytic leukemia |
| M4 | Acute myelomonocytic leukemia |
| M4 eos | Acute myelomonocytic leukemia with |
| | eosinophilia |
| M5 | Acute monocytic leukemia |
| M6 | Acute erythroid leukemia |
| M7 | Acute megakaryoblastic leukemia |

The present invention provides methods for treating AML and the use of arginine-depleting agents for the manufacture of a medicament for the treatment of AML of any subtype. In some embodiments, the invention provides methods, and the use of arginine-depleting agents for the manufacture of medicaments for treating FAB AML M0. The methods and medicaments of the present invention may also be used to treat FAB AML M2. In other embodiments, the invention provides methods and medicaments for treating FAB AML M4. In further embodiments, the invention provides methods and medicaments for treating FAB AML M4 eos. In still further embodiments, the invention provides methods and medicaments for treating FAB AML M5. Methods and medicaments for treating FAB AML M6 are also provided herein. The present invention also provides methods and medicaments for treating FAB AML M7. Although the methods and medicaments provided herein are presented in the context of treating AML subtypes as defined by the FAB classification system, the skilled person will understand that they may be used to treat cases of AML classified using any other system or method of classification.

The FAB AML classification system was established in 1976 and is well known in the art. A person skilled in the art can easily identify the FAB AML subtype of a sample using, for example, histochemical staining and microscopy. The AML sample used may be obtained, for example, from peripheral blood, a bone marrow aspirate or a biopsy. A detailed description of each FAB AML subtype, including images to aid identification, is provided in Charles A. Schiffer, MD and Richard M. Stone, MD (2003) in “Holland-Frei Cancer Medicine, 6th edition”, Kufe D W, Pollock R E, Weichselbaum R R, et al. (eds.) Hamilton (ON), 1983.

Arginine is required for a variety of metabolic pathways. Many tumours are auxotrophic for arginine due to low levels or the absence of argininosuccinate synthetase (ASS) and/or ornithine transcarbamoylase (OTC), which are required for arginine synthesis. In most cases of AML, the cells are deficient in ASS1, the gene encoding ASS in humans. It would be easy for a person skilled in the art to determine whether the cells from an AML sample were deficient in one or both of the aforementioned enzymes using well-known methods such as Western Blot, ELISA SDS-PAGE or immunoprecipitation.

Some embodiments of the present invention provide methods for treating AML in a subject comprising administering a therapeutically effective amount of an arginine-depleting agent to the subject. Further embodiments provide the use of arginine-depleting agents for the manufacture of a medicament for the treatment of AML in a subject in need thereof. The subject may be any animal (e.g., a mammal), including, but not limited to, humans, non-human primates, canines, felines, and rodents.

Arginine-Depleting Agents

An arginine-depleting agent used in the treatment methods and medicaments described herein may be any arginine-depleting agent known in the art to be capable of reducing plasma and/or cellular levels of arginine in a subject. The arginine-depleting agent may, for example, be a small molecule or protein.

In some embodiments, the arginine-depleting agent comprises an arginine catabolic enzyme. Non-limiting examples of arginine catabolic enzymes include arginase, arginine deiminase, arginine decarboxylase and arginine 2-monooxygenase.

The arginase may be any arginase known in the art, such as those produced by bacteria, fungi, fish, humans, bovines, swine, rabbits, rodents, primates, sheep and goats. Non-limiting examples of arginases include Bacillus caldovelox arginase, Thermus thermophilus arginase, Capra hircus arginase I, Heterocephalus glaber arginase I, Bos taurus arginase I, Sus scrofa arginase I, Plecoglossus altivelis arginase I, Salmo salar arginase I, Oncorhynchus mykiss arginase I, Osmerus mordax arginase I, Hyriopsis cumingii arginase I, Rattus norvegicus arginase I, Mus musculus arginase I, Homo sapiens (human) arginase I, Pan troglodytes arginase I, Oryctolagus cuniculus arginase I, Rattus norvegicus arginase II, Mus musculus arginase II, Homo sapiens (human) arginase II, Bostaurus arginase II, Heterocephalus glaber arginase II, Pan troglodytes arginase II, Oryctolagus cuniculus arginase II, Delftia arginase, Bacillus coagulans arginase, Hoeflea phototrophica arginase and Roseiflexus castenholzii arginase. Other examples include arginases from Bacillus methanolicus, Bacillus sp. NRRL B-14911, Planococcus donghaensis, Paenibacillus dendritiformis, Desmospora sp., Methylobacter tundripaludum, Stenotrophomonas sp., Microbacterium laevaniformans, Porphyromonas uenonis, Agrobacterium sp., Octadecabacter arcticus, Agrobacterium tumefaciens, Anoxybacillus flavithermus, Bacillus pumilus, Geobacillus thermoglucosidasius, Geobacillus thermoglucosidans, Brevibacillus laterosporus, Desulfotomaculum ruminis, Geobacillus kaustophilus, Geobacillus thermoleovorans, Geobacillus thermodenitrificans, Staphylococcus aureus, Halophilic archaeon DL31, Halopigerxanaduensis, Natrialba magadii, Plasmodium falciparum, Helicobacter pylori, and the like.

An arginine deiminase used in the methods and medicaments of the present invention may be any arginine deiminase known in the art, such as those produced from Mycoplasma, Lactococcus, Pseudomonas, Steptococcus, Escherichia, Mycobacterium or Bacillus microorganisms. Exemplary arginine deiminases include, but are not limited, to those produced by Mycoplasma hominis, Mycoplasma arginini, Mycoplasma arthritidis, Clostridium perfringens, Bacillus licheniformis, Borrelia burgdorferi, Borrelia afzellii, Enterococcus faecalis, Lactococcus lactis, Bacillus cereus, Streptococcus pyogenes, Steptococcus pneumoniae, Lactobacillus sake, Giardia intestinalis, Mycobacterium tuberculosis, Pseudomonas plecoglossicida, Pseudomonas putida, Pseudomonas aeruginosa, and the like.

The arginine decarboxylase may be any arginine decarboxylase known in the art, such as those produced by Escherichia coli., Salmonella typhimurium, Chlamydophila pneumoniae, Methanocaldococcus jannaschii, Paramecium bursaria Chlorella virus 1, Vibrio vulnificus YJ016, Campylobacter jejuni subsp., Trypanosoma cruzi, Sulfolobus solfataricus, Bacillus licheniformis, Bacillus cereus, Carica papaya, Nicotianatobacum, Glycine max, Lotus coniculata, Vibrio vulnificus, Vibrio cholerae, Mus musculus, Thermotoga, Rattus norvegzcus, Homo sapiens, Bos taurus, Susscrofa, Thermus thermophiles, Thermus parvatiensis, Thermus aquaticus, Thermus thermophiles, Thermus islandicus, Arabidopsis thaliana, Avena sativa, and the like.

An arginine 2-monooxygenase used in the methods and medicaments of the present invention may be any arginine 2-monooxygenase known in the art, such as those produced from Arthrobacter globiformis IFO 12137, Arthrobacter simplex IFO 12069, Brevibacterium helvolum IFO 12073, Helicobacter cinaedi CCUG 18818, Streptomyces griseus, and the like.

The arginine-depleting agents of the present invention may comprise naturally occurring and/or synthetic products. In some embodiments of the invention, the arginine-depleting agents comprise naturally occurring arginine catabolic enzymes. In other embodiments, the arginine-depleting agents comprise synthetic arginine catabolic enzymes.

The arginine-depleting agents may comprise full proteins and/or functional fragments and/or variants thereof. Arginine decarboxylases, arginine deiminases, arginine 2-monooxygenases, arginases and other arginine-depleting agents used in the methods and uses may be modified to improve their pharmacokinetic properties, such as by fusion of proteins and/or functional fragments and/or variants thereof with human serum albumin, an albumin binding domain, an Fe region of an immunoglobulin, a polyethylene glycol (PEG) group, or a combination thereof. In some embodiments of the invention, one or more of the aforementioned modifications lengthens the half-life of the arginine-depleting agent. In further embodiments, the increase in half-life results in a reduction of the frequency of administration of the arginine-depleting agent required to achieve the same outcome. One or more of the aforementioned modifications to the arginine-depleting agents may reduce immunogenicity, which may help to avoid adverse effects.

In some embodiments of the present invention, arginine catabolic enzymes may be engineered to include specific sites on the enzyme where, for example, PEG can be selectively attached. The selected PEGylation sites may be located at a site removed from the active site of the enzyme, and/or may be generally exposed to solvent to allow reaction with PEGylation reagents.

Any PEGylation reagent known in the art can be used to covalently attach PEG to the arginine catabolic enzymes described herein. Exemplary PEGylation reagents include, but are not limited to mPEG-ALD (methoxypolyethylene glycol-propionaldehyde); mPEG-MAL (methoxypolyethylene glycol-maleimide); mPEG-NHS (methoxypolyethylene glycol-N-hydroxy-succinimide); mPEG-SPA (methoxypolyethylene glycol-succinimidyl propionate); and mPEG-CN (methoxypolyethylene glycol-cyanuric chloride).

In some embodiments, the PEG group has a molecular weight of about 5,000 to about 20,000 amu, about 5,000 to about 15,000 amu, about 5,000 to about 12,000 amu, about 7,000 to about 12,000 amu, or about 7,000 to about 10,000 amu. In certain embodiments, the PEG group has a molecular weight of about 2,000 amu to 10,000 amu. In some embodiments, the PEG group is PEG4,000, PEG5,000, PEG6,000, or PEG7,000.

The PEG group may be covalently attached directly to the enzyme or attached via a linker. In certain embodiments, the enzyme is covalently attached via a propionic acid linker to PEG.

Arginine catabolic enzymes may be fused to proteins with an inherently long serum half-life, which may result in more desirable pharmacokinetic profiles. The arginine-depleting agents of the present invention may comprise an antibody Fc domain and/or serum albumin. The arginine-depleting agents may comprise arginine catabolic enzymes genetically fused to an antibody Fc domain and/or serum albumin. In some embodiments, the Fe region of an immunoglobulin is from human immunoglobulin, for example, human IgG. In some embodiments, the enzymes may be fused to an albumin binding domain. In some embodiments, the enzymes may be fused to human transferrin.

In further embodiments of the invention, the arginine-depleting agents comprise arginine catabolic enzymes fused to non-structured polypeptides. Fusion of the enzymes to non-structured polypeptides may increase the overall size and/or hydrodynamic radius of the agents. In some embodiments of the invention, arginine catabolic enzymes are fused to any one or more of XTEN, which is a recombinant PEG mimetic (XTENylation), PAS, which is a proline-alanine-serine polymer (PASylation), ELP, which is an elastin-like polypeptide (ELPylation), HAP, which is a homo-amino-acid polymer (HAPylation), and artificial gelatin-like protein (GLK).

The arginine catabolic enzymes used in some embodiments of the invention may be fused to anionic polypeptides, which may increase the negative charge of the agents. Enzymes may be fused to a carboxy-terminal peptide (CTP). One non-limiting example of suitable CTP fusion is the genetic fusion of the CTP from the human chorionic gonadotropin (CG) β chain.

Arginine catabolic enzymes may be linked to serum albumin via non-covalent interactions with serum albumin, which may also extend the half-life of the agents. In one non-limiting example of an embodiment of the invention, an albumin-binding moiety is either conjugated or genetically fused to the therapeutic enzyme. Many types of moieties can be used, including, but not limited to (i) molecules with intrinsic affinity for albumin; (ii) peptides, antibody fragments, alternative scaffolds, and small chemicals generated and selected to exhibit albumin binding activity.

Recombinant fusion proteins were first used in the 1980s and are created by the fusion of two or more genes which each encode a separate protein. A variety of methods for the synthesis of fusion proteins are well known in the art. See, for example, Yu et al., Biotechnology Advances, 2015; 33: 155-164, which provides a review of the most common approaches currently used for the design and construction of synthetic fusion proteins. Strohl, Biodrugs, 2015; 29(4): 215-239, provides another detailed review of fusion proteins and outlines the advantages and disadvantages of both fusion methods and different types of fusion protein.

In some embodiments of the invention, the arginine-depleting agent comprises or consists of an amino acid sequence having at least 75%, 80%, 85%, 87%, 89%, 90%, 91%, 92%, 93%, 94%, 95%, 96%, 97%, 98%, 99% or 100% sequence identity to SEQ ID NO: 1. In further embodiments, the arginine-depleting agent comprises or consists of an amino acid sequence having at least 95%, 96%, 97%, 98% 99% or 100% sequence identity to SEQ ID NO: 1. In still further embodiments, the arginine-depleting agent comprises or consists of an amino acid sequence as defined in SEQ ID NO: 1.

Methods for assessing the level of homology and identity between sequences are well known in the art. The percentage of sequence identity between two sequences may, for example, be calculated using a mathematical algorithm. A non-limiting example of a suitable mathematical algorithm is described in the publication of Karlin and colleagues (1993, PNAS USA, 90:5873-5877). This algorithm is integrated in the BLAST (Basic Local Alignment Search Tool) family of programs (see also Altschul et al. (1990), J. Mol. Biol. 215, 403-410 or Altschul et al. (1997), Nucleic Acids Res, 25:3389-3402) accessible via the National Center for Biotechnology Information (NCBI) website homepage (https://www.ncbi.nlm.nih.gov). The BLAST program is freely accessible at https://blast.ncbi.nlm.nih.gov/Blast.cgi. Other non-limiting examples include the Clustal (http://www.clustal.org/) and FASTA (Pearson (1990), Methods Enzymol. 83, 63-98; Pearson and Lipman (1988), Proc. Natl. Acad. Sci. U. S. A 85, 2444-2448.) programs. These and other programs can be used to identify sequences which are at least to some level identical to a given input sequence. Additionally or alternatively, programs available in the Wisconsin Sequence Analysis Package, version 9.1 (Devereux et al. 1984, Nucleic Acids Res., 387-395), for example the programs GAP and BESTFIT, may be used to determine the percentage of sequence identity between two polypeptide sequences. BESTFIT uses the local homology algorithm of Smith and Waterman (1981, J. Mol. Biol. 147, 195-197) and identifies the best single region of similarity between two sequences. Where reference herein is made to an amino acid sequence sharing a specified percentage of sequence identity to a reference amino acid sequence, the difference/s between the sequences may arise partially or completely from conservative amino acid substitution/s. In such cases, the sequence identified with the conservative amino acid substitution/s may substantially or completely retain the same biological activity of the reference sequence.

Administration of Arginine-Depleting Agents

For therapeutic use, the arginine-depleting agents described herein may be prepared as pharmaceutical compositions containing a therapeutically effective amount of an arginine-depleting agent described herein as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. As a non-limiting example, 0.9% saline and 0.3% glycine can be used. These solutions may be sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and colouring agents, etc. The concentration of the arginine-depleting agent in such pharmaceutical formulation can vary widely and may be selected based on required dose, fluid volumes, viscosities, etc., according to the particular mode of administration selected. Suitable vehicles and formulations are described, for example, in Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989.

The concentration of plasma arginine in the subject needed to observe a therapeutic effect may vary based on numerous factors, including the condition of the subject and/or the type and severity of the AML and/or diet composition. The selection of the target plasma arginine levels is well within the skill of a person of ordinary skill in the art.

The determination of the duration of treatment e.g., the duration of time the plasma arginine concentrations are maintained in a depleted state in the subject, is well within the skill of a person of ordinary skill in the art. In certain embodiments, the duration of treatment is more than 1 week, more than 2 weeks, more than 3 weeks, more than 4 weeks, more than 5 weeks, more than 6 weeks, more than 7 weeks, more than 8 weeks, more than 9 weeks, more than 10 weeks, more than 11 weeks, more than 12 weeks, more than 24 weeks, more than 28 weeks, more than 32 weeks, more than 36 weeks, more than 40 weeks, more than 44 weeks, more than 48 weeks, more than 52 weeks, or more than 56 weeks.

No limitation applies in relation to the mode of administration of the arginine-depleting agents in the methods and uses of the present invention. In some embodiments of the invention, the mode of administration of the arginine-depleting agents is intravenous. The mode of administration for therapeutic use of the arginine-depleting agents described herein may be any suitable route that delivers the agents to the subject, such as parenteral administration, e.g., intradermal, intramuscular, intraperitoneal, intravenous and/or subcutaneous; pulmonary; transmucosal (e.g., oral, intranasal, intravaginal and/or rectal); using a formulation in a tablet, capsule, solution, suspension, powder, gel and/or particle; and contained in a syringe, an implanted device, osmotic pump, cartridge and/or micropump; or other means appreciated by the skilled artisan, as well known in the art.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to the following specific Examples, which should not be construed as in any way limiting.

### Example 1: Cytotoxicity of NEI-01 in a Range of Cancer Cell Lines

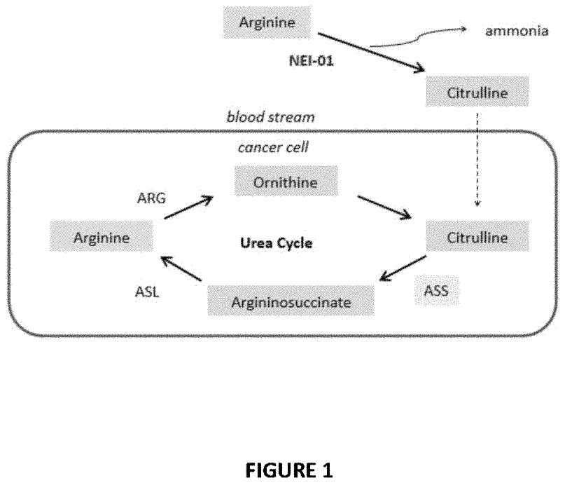

Exogenous arginine is required for growth in some argininosuccinate synthetase (ASS)-deficient cancers. NEI-01 is a recombinant albumin-binding arginine deiminase which can convert arginine to citrulline & ammonia, and inhibits growth in various ASS-deficient cancers by depleting arginine (FIG. 1). Cytotoxicity assay results demonstrated that NEI-01 depleted arginine and inhibited cancer cell growth (especially in the case of ASS1 deficient cancer cell lines) in a range of different cancer cell lines (Table 2).

TABLE 2
Cytotoxicity assay results for different cancer
cell lines following treatment with NEI-01.

| | | | ASS1 | |
|---|---|---|---|---|
| | | | Expression | IC50 |
| Cancer | Cell Line | Origin | (Protein) | (μg/ml) |
| Hepatocellular | Hepa 1-6 | Mouse | − | 0.0080 |
| Carcinoma | Hep-55 1C | Mouse | − | 0.0076 |
| | SK-HEP-1 | Human | − | 0.0292 |
| Colon Cancer | Lovo | Human | + | >10 |
| | COLO 205 | Human | + | >10 |
| | HT-29 | Human | − | 0.1058 |
| Breast Cancer | MCF7 | Human | + | >10 |
| | MDA-MB-231 | Human | − | 0.0168 |
| | 4T1 | Mouse | − | 0.0389 |
| Prostate Carcinoma | 22Rv1 | Human | − | 0.0262 |
| Malignant | A375 | Human | − | 0.0081 |
| Melanoma | | | | |
| Cervical Carcinoma | C-33 A | Human | − | 0.0126 |
| Pancreatic | MIA PaCa-2 | Human | − | 0.0036 |
| Carcinoma | | | | |

The results of cytotoxicity assays for different AML cell lines after treatment with NEI-01 are provided in Table 3.

TABLE 3
Cytotoxicity assay results for different AML
cell lines following treatment with NEI-01.

| | | | ASS1 | | |
|---|---|---|---|---|---|
| AML | | | Expression | IC50 | Maximum |
| subtype | Cell Line | Origin | (Protein) | (ug/ml) | inhibition |
| M0 | KG-1 | Human | − | 0.0033 | 83% |
| M2 | Kasumi-1 | Human | + | 0.0350 | 19% |
| M2 | HL-60 | Human | − | 0.0068 | 73% |
| M5 | AML-193 | Human | − | 0.0034 | 63% |
| M5 | THP-1 | Human | + | 0.2316 | 11% |
| M4 | C1498 | Mouse | − | 0.0031 | 99% |

### Example 2: Apoptosis and Autophagy. NEI-01 Treatment of Pancreatic Cancer Cell Line Mia PaCa-2

To confirm the arginine deprivation-mediated reduction of cell viability by autophagic cell death, Mia PaCa-2 cells were treated with designated concentrations of NEI-01 with or without choloquine (CQ). At indicated time-points, cells were harvested and subjected to immunoblotting using antibodies against several autophagic and apoptotic markers.

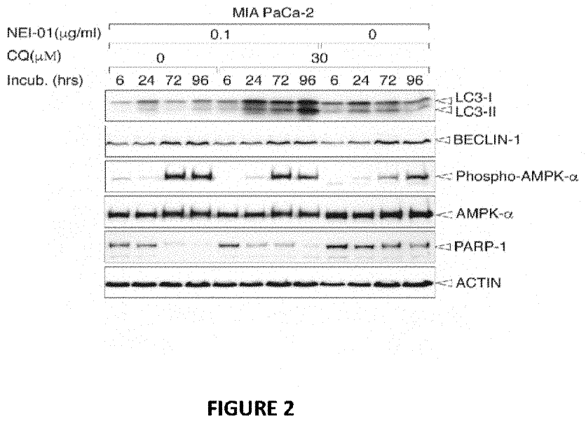

As shown in FIG. 2, the expression levels of autophagic markers LC3-II, BECLIN-1 and phospho-AMPKα were increased upon NEI-01 treatment, suggesting autophagy plays a role in NEI-01-induced cell death. Conversely, expressions levels of apoptotic marker PARP-1 decreased upon NEI-01 treatment, demonstrating the activation of apoptotic pathways. These results show that apoptosis and autophagy play a role in the arginine deprivation-mediated mechanism of cell death.

### Example 3. NEI-01 Treatment of AML Cell Line HL-60

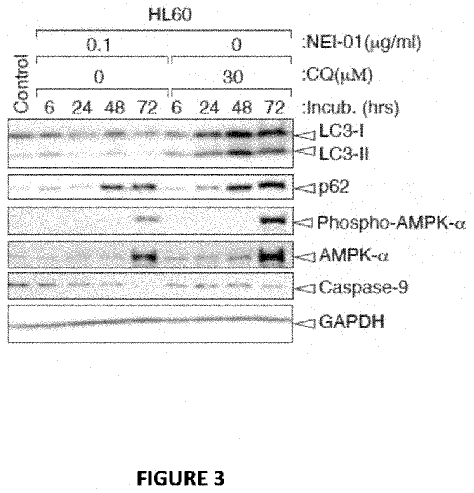

To further confirm the arginine deprivation-mediated reduction of cell viability by autophagic cell death, ASS1-deficient HL-60 AML cells were treated with NEI-01 and CQ. As shown in FIG. 3, expression levels of autophagic markers LC3-II, p62, phospho-AMPKα and AMPKα increased upon NEI-01 treatment, suggesting autophagy plays a role in NEI-01-induced cell death. Conversely, expressions levels of apoptotic marker Caspase-9 decreased following NEI-01 treatment, demonstrating the activation of apoptotic pathways. These results show that apoptosis and autophagy play a role in the arginine deprivation-mediated mechanism of cell death.

### Examples 4 to 10: Effect of NEI-01 on AML Subtypes

Two major classification systems exist for identifying AML subtypes—the French-American-British (FAB) system and the World Health Organization (WHO) classification system. The FAB system is the one most commonly used and will be used herein. According to the FAB system, most people diagnosed with AML have one of nine different kinds (subtypes) of AML: M0, M1, M2, M3, M4, M4 eos, M5, M6 and M7.

### Example 4: Anticancer Activity of NEI-01 in the C1498 Syngeneic Acute Myeloid Leukemia (FAB AML M4) Model

This study aimed to evaluate the anticancer activity of the arginine-depriving enzyme, NEI-01 in a C1498 syngeneic AML (FAB AML M4) Model.

Murine argininosuccinate synthase (ASS1)-deficient C1498 cells co-labeled with luciferase and green fluorescent protein (GFP) were intravenously (i.v.) transplanted into C57BL/6 mice to establish a syngeneic AML model. The mice were randomly divided into 4 groups. Details of the 4 groups and their corresponding treatment regimens are provided in Table 4.

TABLE 4
Groups and treatment regimens for C1498 Syngeneic
Acute Myeloid Leukaemia (FAB AML M4) Model study.

| | | No. of | |
|---|---|---|---|
| Group | Treatment Regimen | animals | Duration |
| 1 | PBS, twice a week, i.v. | 7 | 4 weeks |
| 2 | NEI-01 140 U/kg, once a week, i.v. | 8 | |
| 3 | NEI-01 280 U/kg, once a week, i.v. | 9 | |
| 4 | NEI-01 280 U/kg, twice a week, i.v. | 8 | |

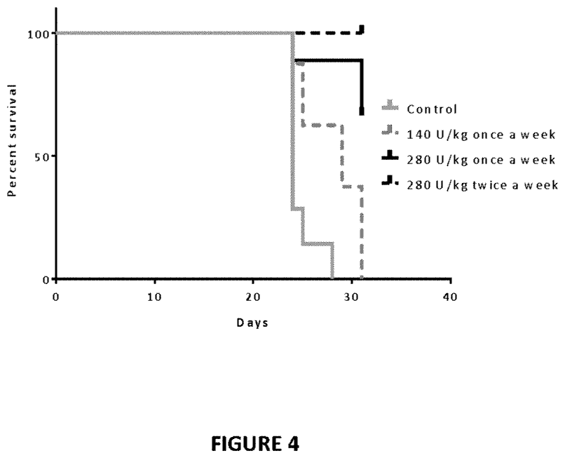

The results showed that treatment with NEI-01 significantly prolonged the overall survival of mice with AML subtype M4 compared with PBS-treated controls (FIG. 4). The median survival day (MSD) was prolonged from 24 days in Group 1 (control group) to 29 days in Group 2 (treated with NEI-01 140 U/kg once a week, p=0.0058 vs control group).

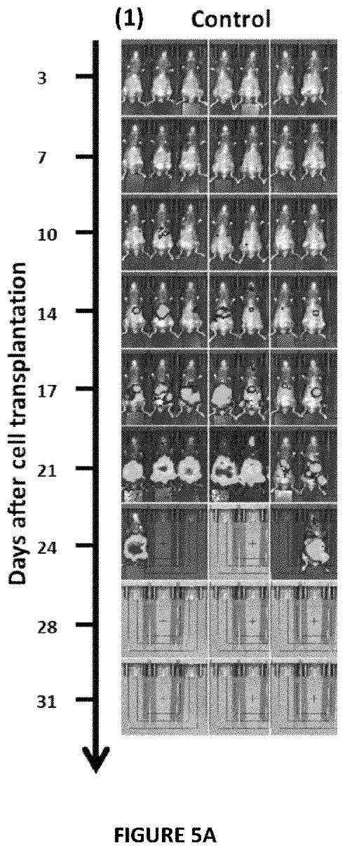

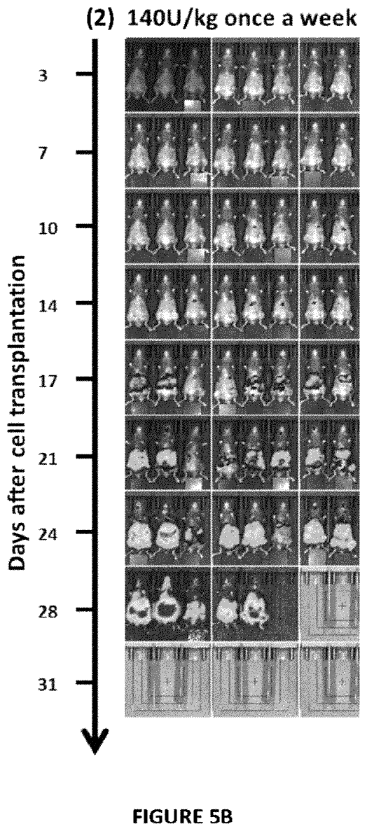

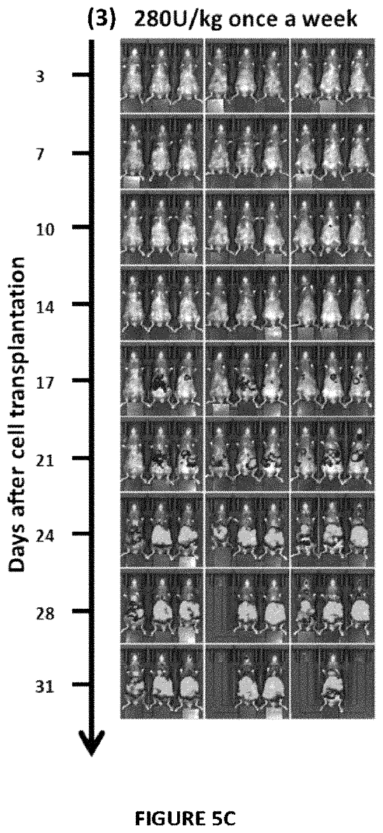

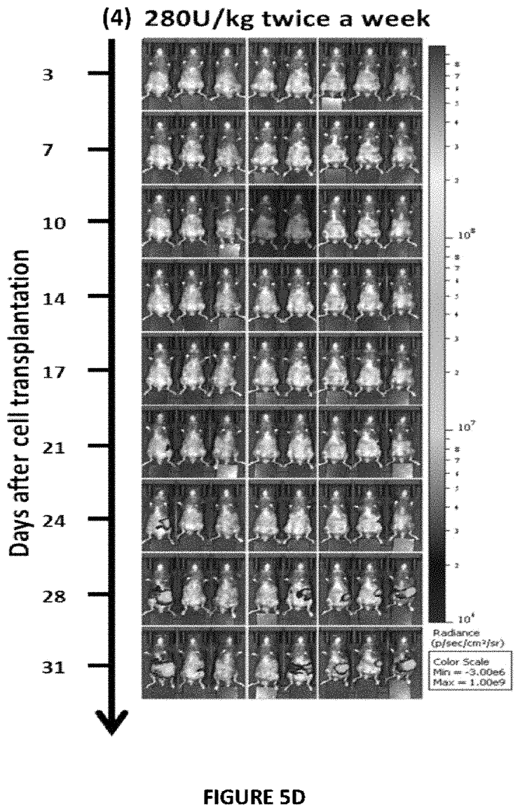

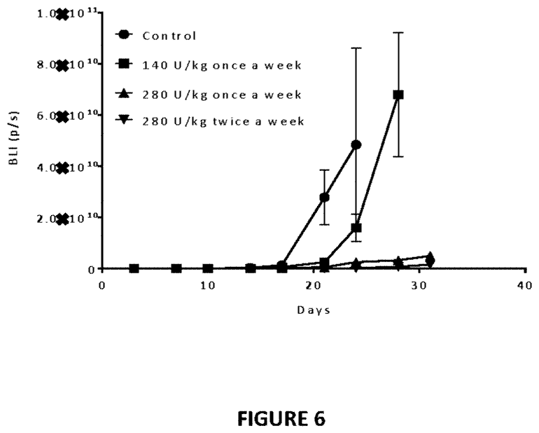

Moreover, more than 60% of the mice in Group 3 (treated with NEI-01 280 U/kg once a week) and all of the mice in Group 4 (treated with NEI-01 280 U/kg twice a week) survived until the end of the experiment. The median survival rate was >31 days in Group 3 and Group 4 (Group 3 vs control, p=0.0003; Group 4 vs control, p<0.0001). Consistent with the results observed for overall survival, treatment with NEI-01 significantly reduced the total leukemia burden in addition to slowing down disease progression (FIGS. 5 and 6). This anticancer activity of NEI-01 was exhibited in a dose-dependence manner.

This study demonstrated a potent anticancer activity of NEI-01 in a C1498 syngeneic AML M4 model.

### Example 5: Anticancer Activity of NEI-01 in a KG-1-Derived Acute Myeloid Leukaemia (FAB AML M0) Xenograft Model

In this study, the anticancer effect of NEI-01 was evaluated in a murine xenograft model.

Human argininosuccinate synthase (ASS1)-deficient M0-subtype acute myeloid leukemia KG-1 cells were subcutaneously injected into immunodeficient BALB/c nude mice. When the tumour volume reached 180 mm³, the mice were intravenously (i.v.) treated with buffer MHT or NEI-01 (280 U/kg) once a week. Details of the study groups and their corresponding treatment regimens are provided in Table 5.

TABLE 5
Groups and treatment regimens for KG-1-Derived Acute
Myeloid Leukaemia (FAB AML M0) Xenograft Model study.

| | | No. of | |
|---|---|---|---|
| Group | Treatment Regimen | animals | Duration |
| 1 | Control, Buffer MHT, once a week, i.v. | 9 | 4 weeks |
| 2 | NEI-01 280 U/kg, once a week, i.v. | 9 | |

The tumour volume was measured every 3 days. After 4 weeks, the mice (n=9) were sacrificed. The xenograft tumours were then dissected and individually weighed.

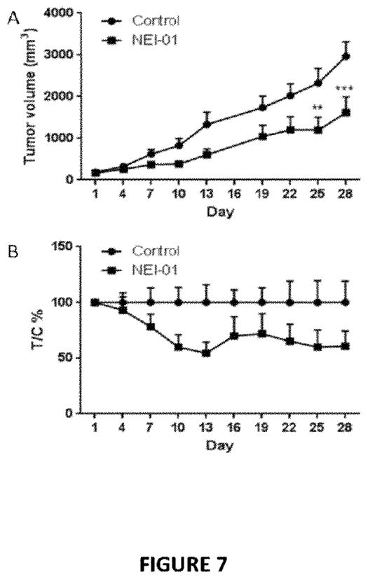

The results showed that NEI-01 treatment significantly reduced the volume of the tumours (FIG. 7). A 39% reduction was observed by Day 28. A final T/C ratio reached at 60.6%. At the termination of the study, the tumour weights were 3.41±0.53 g in the control group and 2.03±0.47 g in the NEI-01 (280 U/kg once a week) treatment group. There was a 40.38% decrease in tumour weight.

This study demonstrated efficient anticancer activity of NEI-01 in murine AML M0 KG-1 xenografts.

### Example 6: Anticancer Activity of NEI-01 in a HL-60 Derived Acute Myeloid Leukemia (FAB AML M2) Orthotopic Model

In this study, the anticancer activity of NEI-01 was evaluated in a murine orthotopic AML model.

Human argininosuccinate synthase (ASS1)-deficient M2-subtype acute myeloid leukemia HL-60 cells co-labeled with luciferase and green fluorescent protein (GFP) were intravenously (i.v.) transplanted into nonobese diabetic/severe combined immunodeficiency (NOD/SCID) mice to establish an orthotopic AML model. The mice were randomly divided into 3 groups. Details of the study groups and their corresponding treatment regimens are provided in Table 6.

TABLE 6
Groups and treatment regimens for HL-60 Derived Acute
Myeloid Leukemia (FAB AML M2) Orthotopic Model study.

| | | No. of | |
|---|---|---|---|
| Group | Treatment Regimen | animals | Duration |
| 1 | PBS, twice a week, i.v. | 10 | 4 weeks |
| 2 | NEI-01 280 U/kg, once a week, i.v. | 10 | |
| 3 | NEI-01 280 U/kg, twice a week, i.v. | 10 | |

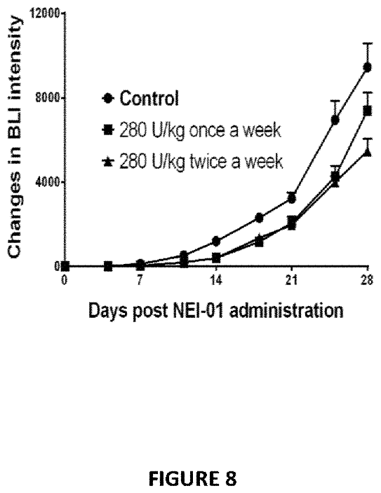

Leukemia cells (HL-60-gfphi-Luc+ cells) were tracked and the total leukemia burden was quantified by in vivo BLI. The disease progression was determined by the changes in BLI intensity. The results are shown in FIG. 8. An aggressive disease progression was found with a strong signal evident throughout the AML mice. This progression was significantly inhibited when the mice were treated with NEI-01 either once a week (p<0.05, from Day 4 to Day 25) or twice a week (p<0.01, whole treatment period).

The results demonstrated that treatment with NEI-01 efficiently depleted arginine from plasma in the mouse, resulting in inhibition of disease progression as well as reduction of tumour burden in hematopoietic tissues (including bone marrow and spleen). Disease progression was significantly inhibited when the mice were treated with NEI-01 either once a week (p<0.05, from Day 4 to Day 25) or twice a week (p<0.01, whole treatment period). Particularly efficient activity was observed in the bone marrow and spleen; the tumour burden was significantly reduced in bone marrow (p<0.0001) and spleen (p<0.005) when the mice were treated with 280 U/kg NEI-01 twice a week.

This study demonstrated a potent anticancer activity of NEI-01 in a HL-60 orthotopic AML M2 model.

### Example 7: Anticancer Activity of NEI-01 in a P31/FUJ-Derived Acute Myeloid Leukemia (FAB AML M5) Xenograft Model

In this study, the anticancer activity of NEI-01 was evaluated in a murine xenograft model.

Human argininosuccinate synthase (ASS1)-deficient M5-subtype acute myeloid leukemia P31/FUJ cells were subcutaneously inoculated in nonobese diabetic/severe combined immunodeficiency (NOD/SCID) mice. When the tumour volume reached 200 mm³, the mice were intravenously (i.v.) treated with buffer MHT or NEI-01 (280 U/kg) once a week. The tumour volume was measured every 3-4 days. After 4 weeks, the mice (n=10) were sacrificed and the xenograft tumours were dissected and individually weighed. Details of the study groups and their corresponding treatment regimens are provided in Table 7.

TABLE 7
Groups and treatment regimens for P31/FUJ-Derived Acute
Myeloid Leukemia (FAB AML M5) Xenograft Model study.

| | | No. of | |
|---|---|---|---|
| Group | Treatment Regimen | animals | Duration |
| 1 | Control, Buffer MHT, once a week, i.v. | 10 | 4 weeks |
| 2 | NEI-01 280 U/kg, once a week, i.v. | 10 | |

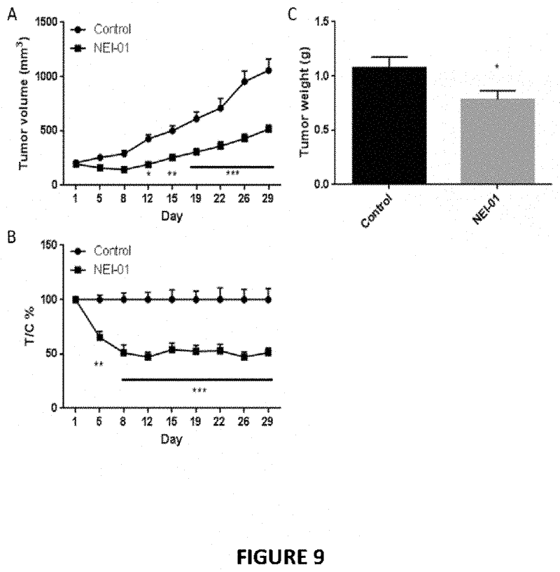

FIG. 9 shows that NEI-01 treatment significantly reduced the tumour volume as well as the tumour weight, resulting in a 51.27% reduction in tumour volume by Day 28. A final T/C ratio reached 51.14%. At the termination of the study, the tumour weights were 1.08±0.98 g in the control group and 0.78±0.08 g in NEI-01 (280 U/kg once a week) treatment group (p<0.05).

This study demonstrated efficient anticancer activity of NEI-01 in murine AML M5 P31/FUJ xenografts.

### Example 8: Anticancer Activity of NEI-01 in a MKPL-1-Derived Acute Myeloid Leukemia (FAB AML M7) Xenograft Model

In this study, the anticancer effect of NEI-01 was evaluated in a murine xenograft model.

Human argininosuccinate synthase (ASS1)-deficient M7-subtype acute myeloid leukemia MKPL-1 cells were subcutaneously inoculated into nonobese diabetic/severe combined immunodeficiency (NOD/SCID) mice. When the tumour size reached 120 mm³, the mice were intravenously (i.v.) treated with buffer MHT or NEI-01 (280 U/kg) once a week. The tumour volume was measured every 2-3 days. After 3 weeks, the mice (n=9) were sacrificed and the xenograft tumours were dissected and individually weighed. Details of the study groups and their corresponding treatment regimens are provided in Table 8.

TABLE 8
Groups and treatment regimens for MKPL-1-Derived Acute
Myeloid Leukemia (FAB AML M7) Xenograft Model study.

| | | No. of | |
|---|---|---|---|
| Group | Treatment Regimen | animals | Duration |
| 1 | Control, Buffer MHT, once a week, i.v. | 9 | 3 weeks |
| 2 | NEI-01 280 U/kg, once a week, i.v. | 9 | |

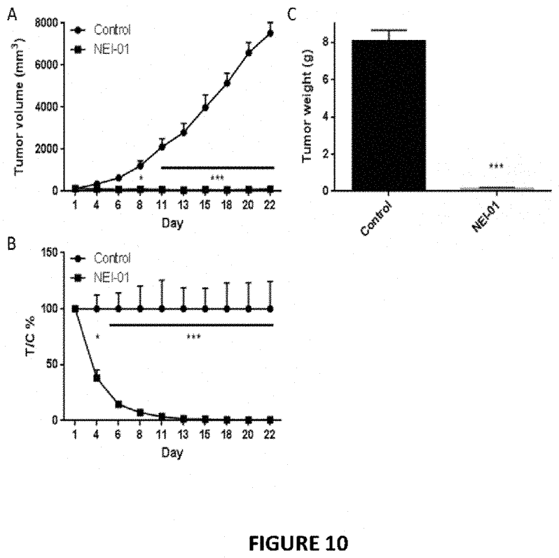

FIG. 10 shows that NEI-01 treatment significantly reduced the tumour volume as well as the tumour weight, resulting in a 99% reduction in tumour volume by Day 22. Tumour weights were 8.05±0.056 g in the control group and 0.15±0.05 g in the NEI-01 (280 U/kg once a week) treatment group. A final T/C ratio reached at 99%.

This study demonstrated efficient anticancer activity of NEI-01 in murine AML M7 MKPL-1 xenografts.

### Example 9: In Vivo Efficacy Study of NEI-01 in the Treatment of a Patient-Derived AM5512 Acute Myeloid Leukemia (FAB AML M7) Model

Patient-derived xenograft (PDX) offers the most translational preclinical model for efficacy screening in cancer drug development. Derived directly from patient tumours and never adapted to grow in vitro, PDX models reflect the heterogeneity and diversity of the human patient population. In this study, the anticancer effect of NEI-01 was evaluated in a Patient-Derived AM5512 (FAB AML M7) Acute Myeloid Leukemia Model.

Human AM5512 cells were intravenously (i.v.) inoculated into nonobese diabetic/severe combined immunodeficiency (NOD/SCID) mice. When the tumour burden in peripheral blood was ˜1.33%, the mice were randomly divided into 3 groups: group 1 (Vehicle), group 2 (NEI-01, 140 U/kg) and group 3 (NEI-01, 280 U/kg) as outlined in Table 9.

TABLE 9
Groups and treatment regimens for Patient-Derived
AM5512 Acute Myeloid Leukemia (FAB AML M7) Model.

| | | | Dose | Route of | | |
|---|---|---|---|---|---|---|
| | | Treat- | level | adminis- | Dosing | Dosing |
| Group | No. | ment | (U/kg) | tration | Frequency | Duration |
| 1 | 10 | Vehicle | — | i.v. | Weekly | on D 1, 8, |
| 2 | 10 | NEI-01 | 140 | | | 15, 22 |
| 3 | 10 | NEI-01 | 280 | | | |

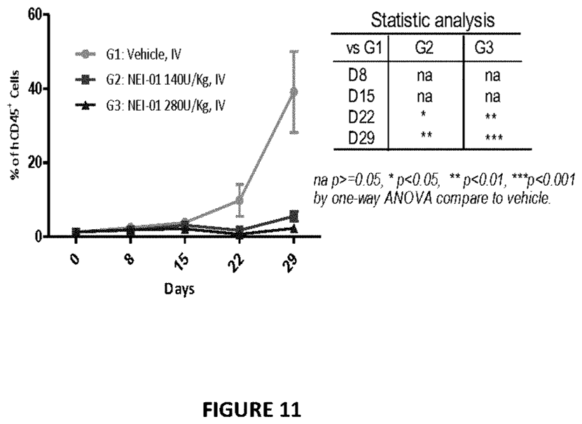

Treatment with NEI-01 (either 280 U/kg or 140 U/kg once a week) significantly inhibited the tumour burden growth in peripheral blood after the 3^{rd} dose of NEI-01 (FIG. 11). At the termination of the study (1 week after the 4th dose of NEI-01), a significant reduction in tumour burden was observed in peripheral blood and hematopoietic tissues (including spleen, liver and bone marrow) following treatment with NEI-01, either 280 U/kg or 140 U/kg once a week (p<0.05, compared to vehicle group). These anti-leukemia effects were exhibited in a dose-dependent manner.

This study provides a strong evidence to support that NEI-01 has a potent anti-leukemia effect in an AM5512 (M7) PDX model.

### Example 10: In Vivo Efficacy Study of NEI-01 in the Treatment of a Patient-Derived AM8096 Acute Myeloid Leukemia (FAB AML M2) Model

In this study, the anticancer effect of NEI-01 was evaluated in a Patient-Derived AM8096 Acute Myeloid Leukemia Model.

Human AM8096 cells were intravenously (i.v.) inoculated into nonobese diabetic/severe combined immunodeficiency (NOD/SCID) mice. When the tumour burden in peripheral blood was ˜1.5%, the mice were randomly divided into 3 groups: group 1 (Vehicle), group 2 (NEI-01, 140 U/kg) and group 3 (NEI-01, 280 U/kg) as outlined in Table 10.

TABLE 10
Groups and treatment regimens for Patient-Derived AM8096
Acute Myeloid Leukemia (FAB AML M2) Model study.

| | | | Dose | Route of | | |
|---|---|---|---|---|---|---|
| | | Treat- | level | adminis- | Dosing | Dosing |
| Group | No. | ment | (U/kg) | tration | Frequency | Duration |
| 1 | 10 | Vehicle | — | i.v. | Weekly | Weekly |
| 2 | 10 | NEI-01 | 140 | | | injection |
| 3 | 10 | NEI-01 | 280 | | | until |
| | | | | | | humane |
| | | | | | | endpoints |
| | | | | | | (on D 1, 8, |
| | | | | | | 15, 22, 29, |
| | | | | | | 36 . . . etc) |

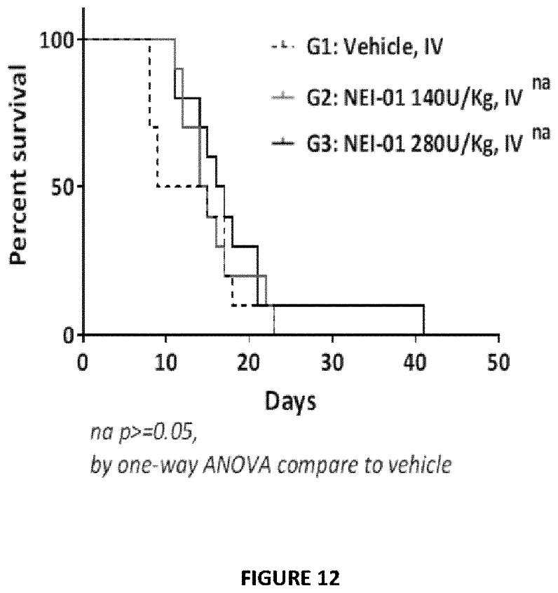

The results showed that treatment with NEI-01 once a week slightly prolonged the median number of survival days from 12 days in the vehicle control group to 14.5 days in the NEI-01 (140 U/kg) treatment group and 16.5 days in the NEI-01 (280 U/kg) treatment group (FIG. 12 and Table 11).

The increase in life-span (ILS) of NEI-01 treated mice (140 U/kg or 280 U/kg) was 20.8% and 37.5% respectively when compared to vehicle controls (Table 11). Excitingly, one of the mice in the NEI-01 (280 U/kg) treatment group survived until 41 days after the initial treatment. These data suggest that NEI-01 has anti-leukemia effects at least in some populations of AM8096 models.

TABLE 11
Median survival days and life-span (ILS)
for each group in the AM8096 model.

| | | | Median | Number of | p value |
|---|---|---|---|---|---|
| | | ILS | survival | remaining at | (VS |
| Group | Treatment | (%) | day (D) | termination | Group1) |
| 1 | Vehicle | — | 12 | None | — |
| 2 | NEI-01, 140 U/kg | 20.8 | 14.5 | None | 0.28 |
| 3 | NEI-01, 280 U/kg | 37.5 | 16.5 | None | 0.12 |

### Example 11: Anticancer Activity of NEI-01 in Jurkat-Derived Leukemia Cancer Xenograft Model

The T-cell immunophenotype of acute lymphoblastic leukemia (T-ALL) accounts for about 15 to 25% of acute leukemia in adults and children. Benefitting from rapid technological advances and an emerging understanding, significant progress has been achieved in the treatment of T-ALL. However, a significant number of patients remain at a high risk for relapse, and few patients survive when the disease recurs. Thus, new therapeutic approaches are urgently needed.

Drug-induced amino acid deprivation is one strategy that has been successfully used in the treatment of acute lymphoblastic leukemia, where asparaginase is an important part of induction chemotherapy. Arginine, as a precursor for initiation of a variety of metabolic pathways, has been confirmed to have a modulatory effect on tumourigenesis. Arginine deprivation has been demonstrated as a promising therapeutic approach against arginine-auxotrophic tumours which lack argininosuccinate synthase (ASS1), a limiting enzyme to synthesize arginine from citrulline. This study aimed to evaluate the anticancer activity of the arginine-depriving enzyme, NEI-01 in a T-ALL Jurkat xenograft model.

Human ASS1-deficient T-ALL Jurkat cells were subcutaneously inoculated into immunodeficient BALB/c nude mice. When the tumour volume reached around 40 mm³, the mice were randomly divided into two groups: a control and NEI-01 treatment group, as outlined in Table 12. The mice were intraperitoneally (i.p.) administered with PBS or NEI-01 (5 U per mouse, ˜280 U/kg) twice a week for 4 weeks. The tumour volume was measured twice a week.

TABLE 12
Groups and treatment regimens for Jurkat-Derived
Leukemia Cancer Xenograft Model study.

| | | No. of | |
|---|---|---|---|
| Group | Treatment Regimen | animals | Duration |
| 1 | PBS, twice a week, i.p. | 3 | 4 weeks |
| 2 | NEI-01 5 U per mouse (~280 U/kg), | 3 | |
| | twice a week, i.p. | | |

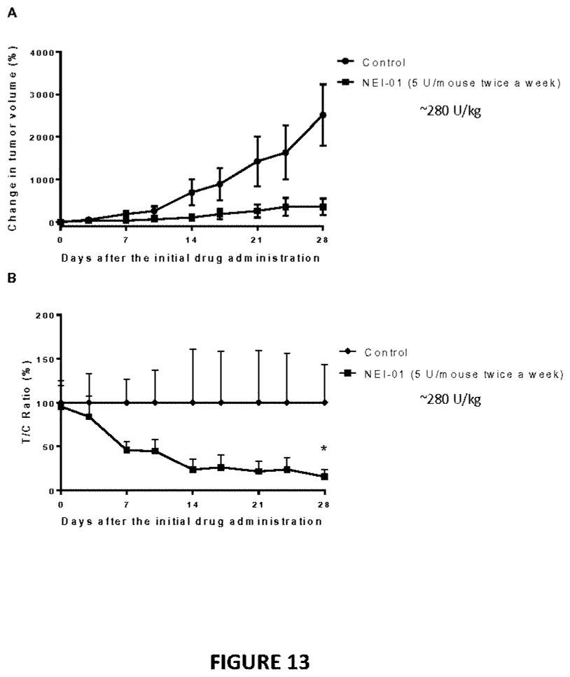

The results showed that treatment with NEI-01 (5 U per mouse, ˜280 U/kg) twice a week significantly inhibited (p≤0.05) the tumour growth when compared with the control group on Day 28 (FIG. 13).

These data provide support for the potent anticancer activity of NEI-01 in a Jurkat-derived leukemia subcutaneous xenograft model.

### Example 12: Determination of NEI-01 in Mice Plasma from Repeated Dose Study

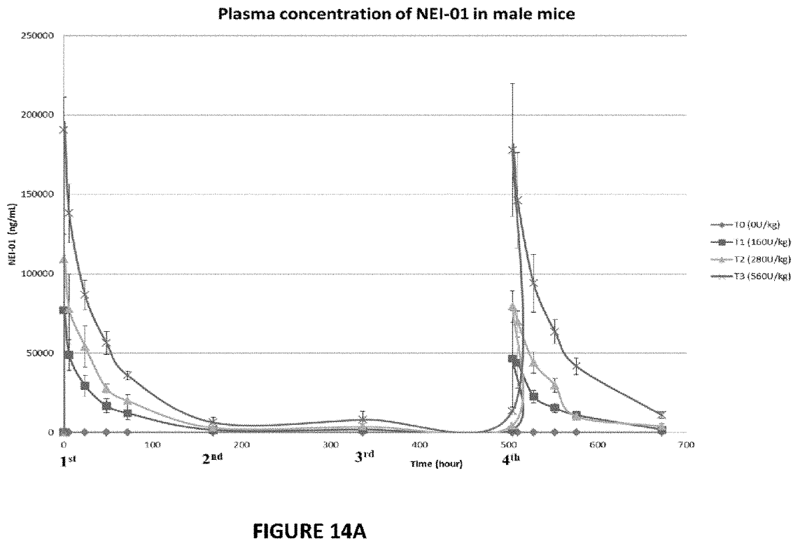

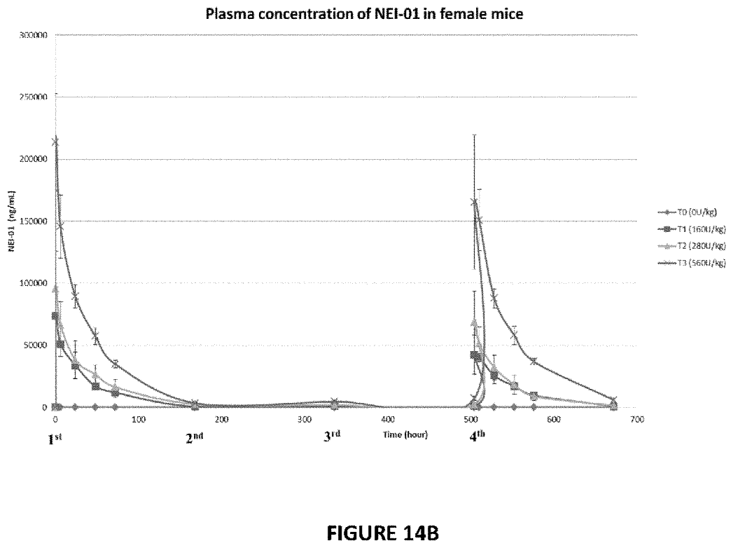

NEI-01 was administered to ICR mice by intravenously once per week for 4 weeks at dosages of 160, 280 and 560 U/kg. Blood samples were taken on Day 1 and Day 22 at pre-dose (−1), 0.25, 6, 24, 48 and 72 h post-dose for all groups on Day 1, pre-dose (−1) for all groups on Day 8 (Week 2), pre-dose (−1) for all groups on Day 15 (Week 3), pre-dose (−1), 0.25, 6, 24, 48 and 72 h post-dose on Day 22 (Week 4) and before sacrificing the mice on Day 29 (Week 5). 5 animals/group/sex/time point and plasma concentrations were quantified (FIG. 14).

The parameters for the pharmacokinetic assessment of NEI-01 for the treatment groups and the results obtained are presented in Table 13 (Day 1) and Table 14 (Day 22). All plasma concentrations of NEI-01 in the vehicle control group were below the limit of quantification. Thus, the vehicle control group data are not presented in the tables.

TABLE 13
Pharmacokinetic parameters and measurements
for Day 1 Mice for NEI-01 treatment groups.

| | 160 U/kg | | 280 U/kg | | 560 U/kg | |
|---|---|---|---|---|---|---|
| | Male | Female | Male | Female | Male | Female |
| Cₘₐₓ^{a} (ng/ml) | 77054 | 73787 | 108986 | 95395 | 190556 | 213700 |
| Tₘₐₓ^{b} (h) | 0.25 | 0.25 | 0.25 | 0.25 | 0.25 | 0.25 |
| T_{1/2}^{c} (h) | 21.04 | 31.90 | 36.58 | 25.87 | 35.71 | 34.37 |
| AUC_{0-72 h}^{d} | 1.98 × 10⁶ | 2.08 × 10⁶ | 3.30 × 10⁶ | 2.70 × 10⁶ | 5.82 × 10⁶ | 6.04 × 10⁶ |
| (ng · h/ml) | | | | | | |

^{a}Cₘₐₓ: maximum NEI-01 concentration
^{b}Tₘₐₓ: time at which Cₘₐₓ occurs
^{c}T_{1/2}: half-life, time taken for Cₘₐₓ to drop in half
^{d}AUC_{0-72 h}: the area under the curve in a plot of drug concentration versus time from time of drug administration (time “0” to time “72 h”)

TABLE 14
Pharmacokinetic parameters and measurements
for Day 22 Mice for NEI-01 treatment groups.

| | 160 U/kg | | 280 U/kg | | 560 U/kg | |
|---|---|---|---|---|---|---|
| | Male | Female | Male | Female | Male | Female |
| Cₘₐₓ^{a} (ng/ml) | 46607 | 42417 | 79378 | 68834 | 177988 | 165310 |
| Tₘₐₓ^{b} (h) | 0.25 | 0.25 | 0.25 | 0.25 | 0.25 | 0.25 |
| T_{1/2}^{c} (h) | 26.55 | 39.51 | 31.82 | 26.82 | 37.38 | 32.20 |
| AUC_{0-72 h}^{d} | 1.65 × 10⁶ | 1.67 × 10⁶ | 2.83 × 10⁶ | 2.04 × 10⁶ | 6.27 × 10⁶ | 5.97 × 10⁶ |
| (ng · h/ml) | | | | | | |

^{a}Cₘₐₓ: maximum NEI-01 concentration
^{b}Tₘₐₓ: time at which Cₘₐₓ occurs
^{c}T_{1/2}: half-life, time taken for Cₘₐₓ to drop in half
^{d}AUC_{0-72 h}: the area under the curve in a plot of drug concentration versus time from time of drug administration (time “0” to time “72 h”)

Following intravenous administration of NEI-01 to mice, systemic exposure to NEI-01 was observed and the mean value Tₘₐₓ was 0.25 h post-dose in both males and females on Day 1. The T_{1/2} was between 21.04 and 36.58 hours.

Systemic exposure (as measured by AUC₀₋₇₂) to NEI-01 increased with dose in a proportional manner in males and females on both Day 1 and Day 22. The AUC₀₋₇₂ was similar in males and females on Day 1 (5.82×10⁶ ng·h/ml ˜6.04×10⁶ ng·h/ml) and Day 22 (5.97×10⁶ ng·h/ml ˜6.27×10⁶ ng·h/ml) at 560 U/kg. Also, C. results were similar to AUC₀₋₇₂ in that results were similar for males and females on both Day 1 and Day 22. There was also no significant difference in body weight between males and female treatment groups.

### INDUSTRIAL APPLICABILITY

The objective of the presently claimed invention is to provide alternative methods for treating AML.

1. A method for treating acute myeloid leukemia (AML) in a subject in need thereof, said method comprising administering a therapeutically effective amount of an arginine-depleting agent to the subject, wherein the AML is of the French-American-British (FAB) subtype M0 (undifferentiated acute myeloblastic leukemia), M2 (acute myeloblastic leukemia with maturation), M4 (acute myeloblastic leukemia with maturation), M4 eos (acute myelomonocytic leukemia with eosinophilia), M5 (acute monocytic leukemia), M6 (acute erythroid leukemia) or M7 (acute megakaryoblastic leukemia).

2. The method according to claim 1, wherein the arginine-depleting agent comprises an arginine catabolic enzyme.

3. The method according to claim 2, wherein the arginine catabolic enzyme is an arginine deiminase, arginase, arginine decarboxylase or arginine 2-monooxygenase.

4. The method according to claim 1, wherein the arginine-depleting agent is a synthetic arginine-depleting agent.

5. The method according to claim 1, wherein the arginine-depleting agent comprises human serum albumin, an albumin binding domain, an Fe region of an immunoglobulin, a polyethylene glycol (PEG) group, human transferrin, XTEN, a proline-alanine-serine polymer (PAS), an elastin-like polypeptide (ELP), a homo-amino-acid polymer (HAP), artificial gelatin-like protein (GLK), a carboxy-terminal peptide (CTP), or a combination thereof.

6. The method according to claim 1, wherein the arginine-depleting agent comprises human serum albumin, an albumin binding domain, or a combination thereof.

7. The method according to claim 1, wherein the arginine-depleting agent comprises or consists of an amino acid sequence having at least 95%, 96%, 97%, 98% or 99% sequence identity to SEQ ID NO: 1.

8. The method according to claim 1, wherein the arginine-depleting agent comprises or consists of an amino acid sequence as defined in SEQ ID NO: 1.

9. The method according to claim 1, wherein the AML is of the FAB subtype M4 or M7.

10. The method according to claim 9, wherein the arginine-depleting agent comprises or consists of an amino acid sequence having at least 95%, 96%, 97%, 98% or 99% sequence identity to SEQ ID NO: 1.

11. The method according to claim 9, wherein the arginine-depleting agent comprises or consists of an amino acid sequence as defined in SEQ ID NO: 1.

12. The method according to claim 1, wherein the AML is auxotrophic for arginine.

13. The method according to claim 1, wherein the arginine-depleting agent is administered intramuscularly, intravenously, subcutaneously or orally.

14. The method according to claim 1, wherein the arginine-depleting agent is administered intravenously.

15. The method according to claim 1, wherein the subject is human.


SEQUENCE LISTING (1 sequence)

SEQ ID NO: 1 - protein, 469 aa, Artificial Sequence
  Synthetic
MLAEAKVLANRELDKYGVSDYYKNLINNAKTVEGVKALIDEILAALPSGSNNNNNNGSGGSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW